(19) United States
(12) Patent Application Publication
(10) Pub. No.: US 2026/0341422 A1
(43) Pub. Date: Oct. 29, 2026

(54) SYSTEM AND METHOD FOR PREDICTING DRUG RESPONSE OF BIOLOGICAL COMPONENT TO SPECIFIC DRUG

(71) Applicant: GRAPHEN DRUGOMICS, INC., New York, NY (US)

(72) Inventors: Sing-Han HUANG, Taipei City (TW); Tzu-Ying TU, Taipei City (TW); Yi-Ting CHEN, Taipei City (TW); Xiang-Yu LIN, Taipei City (TW); Ssu-Ting LIEN, Taipei City (TW); Chiao-Wen CHEN, Taipei City (TW); Cheng-Tang WANG, Taipei City (TW); Ching-Yung LIN, Scarsdale, NY (US)

(21) Appl. No.: 19/661,000

(22) Filed: Apr. 28, 2026

Related U.S. Application Data
(60) Provisional application No. 63/796,479, filed on Apr. 29, 2025.

(51) Int. Cl. G16C 20/30; G16C 20/50; G16C 20/70

(52) U.S. Cl. CPC G16C 20/30; G16C 20/50; G16C 20/70

(57) ABSTRACT

A system includes an encoder, a decoder, an integration component, a signaling-pathway component, an interaction-modeling component and an output component. The encoder constructs a structure diagram based on a line notation of a chemical structure of a specific drug, and performs multi-layer convolution and global add pooling on the structure diagram to obtain a drug-structure vector. The decoder decodes the drug-structure vector into a transcriptomic-response fingerprint vector. The integration component integrates the drug-structure vector and the transcriptomic-response fingerprint vector into a multidimensional vector. The signaling-pathway component, for each cell sample, converts multi-omics data related to the cell sample into a signaling-pathway feature vector. The interaction-modeling component determines an interaction vector based on the multidimensional vector and the signaling-pathway feature vectors using multi-head attention mechanism. The output component integrates the multidimensional vector and the interaction vector into a prediction indicator.

### CROSS-REFERENCE TO RELATED APPLICATION

This application claims the benefit of U.S. Provisional Patent Application No. 63/796,479, filed on Apr. 29, 2025, the entire disclosure of which is incorporated by reference herein.

### FIELD

The disclosure relates to a system and a method for predicting a drug response of a biological component to a specific drug.

### BACKGROUND

A conventional approach to predict a drug response of a biological component (i.e., any living, organic, or functional part of a biological system, ranging from subcellular organelles, e.g., mitochondria, and molecules, e.g., enzymes, to cells, tissues, organisms, and so on) to a specific drug heavily relies on data that is related to gene expression at the cellular level. Even though the conventional approach sometimes may take into account a chemical structure of the specific drug or data that is related to biological signaling pathways, or may use technologies of statistical modeling and deep learning, the conventional approach fails to include the utilization of cross-modal integration, and cannot derive the potential drug response directly based on the chemical structure of the specific drug. In addition, the conventional approach is poor in its generalization ability, and thus the accuracy of prediction made by using the conventional approach may severely drop when the chemical structure of the specific drug is novel. Moreover, the conventional approach is insufficient in interpretability on biological mechanisms due to overemphasizing the accuracy of prediction and neglecting intermediate biological processes affected by the specific drug.

### SUMMARY

Therefore, an object of the disclosure is to provide a system and a method for predicting a drug response of a biological component to a specific drug that can alleviate at least one of the drawbacks of the prior art.

According to one aspect of the disclosure, the system includes a feature analysis module and a response prediction module.

The feature analysis module includes a drug-structure encoder and a transcriptomic-response decoder.

The drug-structure encoder is configured to obtain a line notation describing a chemical structure of the specific drug, to construct a two-dimensional molecular-structure diagram based on the line notation of the chemical structure of the specific drug, and to perform multi-layer convolution and global add pooling on the two-dimensional molecular-structure diagram so as to obtain a drug-structure feature-embedding vector.

The transcriptomic-response decoder is configured to obtain the drug-structure feature-embedding vector from the drug-structure encoder, and to decode the drug-structure feature-embedding vector into a transcriptomic-response fingerprint vector representing transcriptomic response of the biological component to the specific drug.

The response prediction module includes a drug-feature integration component, a signaling-pathway component, an interaction-modeling component and an output component.

The drug-feature integration component is configured to obtain the drug-structure feature-embedding vector, to obtain the transcriptomic-response fingerprint vector from the transcriptomic-response decoder, and to integrate the drug-structure feature-embedding vector and the transcriptomic-response fingerprint vector into a multidimensional vector representing various features of the specific drug.

The signaling-pathway component is configured, for each cell sample related to the biological component, to obtain multi-omics data related to the cell sample and to convert the multi-omics data into a signaling-pathway feature vector, the signaling-pathway feature vector including multiple elements that are related respectively to multiple biological signaling pathways all related to the cell sample.

The interaction-modeling component is configured to obtain the multidimensional vector from the drug-feature integration component and to obtain the signaling-pathway feature vectors that correspond respectively to multiple cell samples related to the biological component from the signaling-pathway component, and to determine an interaction vector representing interaction of the specific drug with each of the biological signaling pathways based on the multidimensional vector and the signaling-pathway feature vectors using multi-head attention mechanism.

The output component is configured to obtain the multidimensional vector from the drug-feature integration component and the interaction vector from the interaction-modeling component, and to integrate the multidimensional vector and the interaction vector into a prediction indicator which indicates the drug response of the biological component to the specific drug.

According to another aspect of the disclosure, the method is to be implemented by a system that includes a drug-structure encoder, a transcriptomic-response decoder, a drug-feature integration component, a signaling-pathway component, an interaction-modeling component and an output component. The method includes steps of:
constructing, by the drug-structure encoder, a two-dimensional molecular-structure diagram based on a line notation describing a chemical structure of the specific drug;
performing, by the drug-structure encoder, multi-layer convolution and global add pooling on the two-dimensional molecular-structure diagram so as to obtain a drug-structure feature-embedding vector;
decoding, by the transcriptomic-response decoder, the drug-structure feature-embedding vector into a transcriptomic-response fingerprint vector representing transcriptomic response of the biological component to the specific drug;
integrating, by the drug-feature integration component, the drug-structure feature-embedding vector and the transcriptomic-response fingerprint vector into a multidimensional vector representing various features of the specific drug;
for each cell sample related to the biological component by the signaling-pathway component,
   obtaining multi-omics data related to the cell sample,
   converting the multi-omics data into a signaling-pathway feature vector, the signaling-pathway feature vector including multiple elements that are related respectively to multiple biological signaling pathways all related to the cell sample;
determining, by the interaction-modeling component, an interaction vector representing interaction of the specific drug with each of the biological signaling pathways based on the multidimensional vector and the signaling-pathway feature vectors using multi-head attention mechanism; and
integrating, by the output component, the multidimensional vector and the interaction vector into a prediction indicator which indicates the drug response of the biological component to the specific drug.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a block diagram illustrating a system for predicting a drug response of a biological component to a specific drug according to an embodiment of the disclosure.

FIGS. 2 to 6 are flow charts cooperatively illustrating a method for predicting a drug response of a biological component to a specific drug according to an embodiment of the disclosure.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

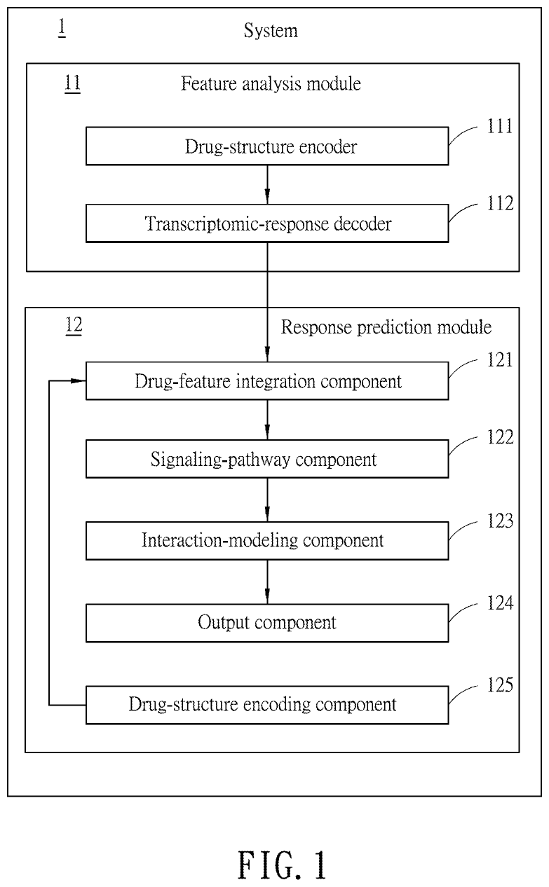

Referring to FIG. 1, an embodiment of a system 1 for predicting a drug response of a biological component to a specific drug according to the disclosure is illustrated. The biological component is any living, organic, or functional part of a biological system, ranging from subcellular organelles (e.g., mitochondria) and molecules (e.g., enzymes) to cells, tissues, organisms, and so on. In one embodiment, the specific drug is exemplarily methotrexate (MTX, which is an antimetabolite), but is not limited thereto.

The system 1 includes a feature analysis module 11 and a response prediction module 12 that are coupled to each other.

The feature analysis module 11 includes a drug-structure encoder 111, and a transcriptomic-response decoder 112 that is coupled to the drug-structure encoder 111.

The drug-structure encoder 111 is a graph isomorphism network (GIN). The drug-structure encoder 111 is configured to obtain a line notation describing a chemical structure of the specific drug. The line notation of the chemical structure of the specific drug follows a specification of simplified molecular input line entry system (SMILES), but is not limited thereto. Since the specification of SMILES has been well known to one skilled in the relevant art, detailed explanation of the same is omitted herein for the sake of brevity. The drug-structure encoder 111 may receive the line notation of the chemical structure of the specific drug based on user input via a keyboard (not shown) of the system 1, or may obtain the line notation of the chemical structure of the specific drug by reading a file that is stored in a storage device (e.g., random access memory, RAM, double data rate synchronous dynamic random access memory, DDR SDRAM, read only memory, ROM, programmable ROM, PROM, flash memory, a hard disk drive, HDD, a solid state disk, SSD, electrically-erasable programmable read-only memory, EEPROM, or any other volatile/non-volatile memory devices, but is not limited thereto) and that records the line notation of the chemical structure of the specific drug. However, the way of obtaining the line notation of the chemical structure of the specific drug is not limited to that disclosed herein and may vary in other embodiments.

The drug-structure encoder 111 is further configured to construct a two-dimensional molecular-structure diagram based on the line notation of the chemical structure of the specific drug. In some embodiments, the drug-structure encoder 111 constructs the two-dimensional molecular-structure diagram by using structure diagram generation (SDG) algorithms, but is not limited thereto. Specifically, the drug-structure encoder 111 is configured to construct the two-dimensional molecular-structure diagram by creating a node matrix for atoms of a molecule of the specific drug by using one-hot encoding, creating an edge matrix for chemical bonds of the molecule of the specific drug, and constructing the two-dimensional molecular-structure diagram based on the node matrix and the edge matrix. It is worthy of note that the node matrix is a stack of feature vectors corresponding respectively to the atoms, and the edge matrix is a set of indexes each corresponding to one of the chemical bonds between two of the atoms. It is worthy of note that use of the open-source toolkit “RDKit” is involved in constructing the two-dimensional molecular-structure diagram. After that, the drug-structure encoder 111 is further configured to perform multi-layer convolution and global add pooling on the two-dimensional molecular-structure diagram so as to obtain a drug-structure feature-embedding vector.

In mathematics, the two-dimensional molecular-structure diagram G_{d} can be expressed as G_{d}=(V_{d}, E_{d}), where V_{d} represents the node matrix, E_{d} represents the edge matrix. During multi-layer convolution, for a current node i that represents one of the atoms of a molecule of the specific drug and each neighbor node j of the current node i, a formula for updating a hidden representation of the current node i under an l^{th} layer of the GIN can be expressed as

$${h}_{i}^{l + 1} = {MLP}^{l} ( {h}_{i}^{l} + {\sum }_{j ∈ N ( i )} {h}_{j}^{l} )$$

where

$${h}_{i}^{l}$$

represents a hidden representation of the current node i under the l^{th} layer of the GIN,

$${h}_{j}^{l}$$

represents the hidden representation of said each neighbor node j under the l^{th} layer of the GIN, MLP^{l} represents a multilayer perceptron to perform local nonlinear transformation for the current node i and said each neighbor node j under the l^{th} layer of the GIN, and

$${h}_{i}^{l + 1}$$

represents the updated hidden representation of the current node i under the l+1^{th} layer of the GIN, l is a positive integer. In this embodiment, l ranges from one to three, but is not limited thereto. After global add pooling has been completed, the drug-structure feature-embedding vector z_{d} can be expressed as

$${z}_{d} = POOL ( \{ {h}_{i}^{l} \vert i ∈ {V}_{d} \} ) ,$$

where

$${h}_{i}^{L}$$

represents the updated hidden representation of the current node i under a final layer of the GIN, i.e., the hidden representation of the current node i that has been updated for three times during multi-layer convolution, and POOL represents a function of global add pooling. Since the multilayer perceptron to perform local nonlinear transformation and the function of global add pooling have been well known to one skilled in the relevant art, detailed explanation of the same is omitted herein for the sake of brevity.

The transcriptomic-response decoder 112 is implemented to be a multilayer perceptron (MLP) that includes a linear layer, a function of layer normalization, a rectified linear unit (ReLU) and a function of random dropout. The transcriptomic-response decoder 112 is configured to obtain the drug-structure feature-embedding vector from the drug-structure encoder 111, and to decode the drug-structure feature-embedding vector into a transcriptomic-response fingerprint vector representing transcriptomic response of the biological component to the specific drug. The transcriptomic-response decoder 112 is trained in advanced to learn the way of mapping the drug-structure feature-embedding vector to the transcriptomic-response fingerprint vector by using training data labeled with gene expression profiling (e.g., a matrix having elements each of which represents a signature value of one gene in response to one drug). Since implementations of the GIN and the MLP have been well known to one skilled in the relevant art, detailed explanation of the same is omitted herein for the sake of brevity.

In mathematics, the transcriptomic-response fingerprint vector ŷ_{d} can be expressed as ŷ_{d}=f_{dec}(z_{d}), where f_{dec}( ) represents a decoding function of the transcriptomic-response decoder 112. In this embodiment, ŷ_{d}∈R^{N}^{_{g}}, where N_{g} represents 978 landmark genes in the L1000 dataset of the NIH LINCS program. The transcriptomic-response decoder 112 is pre-trained by using a loss function

$${ℒ}_{mech} = {‖ {\hat{y}}_{d} - {y}_{d} ‖}_{2}^{2} + λ \cdot ( 1 - cos ( {\hat{y}}_{d} , {y}_{d} ) ) ,$$

where λ is a weight coefficient, ŷ_{d} represents a predicted transcriptomic-response fingerprint vector, and y_{d} represents an actual transcriptomic-response fingerprint vector. It is worthy of note that the loss function takes both numerical errors and cosine similarity into account.

The response prediction module 12 is a multimodal neural network. The response prediction module 12 includes a drug-structure encoding component 125, a drug-feature integration component 121 that is coupled to the drug-structure encoding component 125, a signaling-pathway component 122 that is coupled to the drug-feature integration component 121, an interaction-modeling component 123 that is coupled to the signaling-pathway component 122, and an output component 124 that is coupled to the interaction-modeling component 123.

The drug-structure encoding component 125 of the response prediction module 12 is similar to the drug-structure encoder 111 of the feature analysis module 11, and is implemented to be a GIN. The drug-structure encoding component 125 is configured to obtain the line notation describing the chemical structure of the specific drug, to construct the two-dimensional molecular-structure diagram based on the line notation describing the chemical structure of the specific drug, and to perform multi-layer convolution and global add pooling on the two-dimensional molecular-structure diagram so as to obtain the drug-structure feature-embedding vector.

The drug-feature integration component 121 is configured to obtain the drug-structure feature-embedding vector from the drug-structure encoding component 125, and to obtain the transcriptomic-response fingerprint vector from the transcriptomic-response decoder 112. In some embodiments, the drug-structure encoding component 125 is omitted from the response prediction module 12, and the drug-feature integration component 121 is configured to obtain the drug-structure feature-embedding vector from the drug-structure encoder 111 of the feature analysis module 11. Then, the drug-feature integration component 121 is configured to integrate the drug-structure feature-embedding vector and the transcriptomic-response fingerprint vector into a multidimensional vector representing various features of the specific drug. It is worthy of note that the multidimensional vector is a vector of multiple vectors. Specifically, the drug-feature integration component 121 is configured to obtain the multidimensional vector by projecting the transcriptomic-response fingerprint vector to an embedding dimension consistent with the drug-structure feature-embedding vector via linear transformation to obtain a projection vector, concatenating the drug-structure feature-embedding vector and the projection vector into a concatenated vector, and performing fusion conversion on the concatenated vector to obtain the multidimensional vector.

In mathematics, the multidimensional vector ha can be expressed as h_{d}=f_{fusion}([z_{d}∥g(ŷ_{d})]), where g( ) is a linear transformation for projection, ∥ represents an operation of vector concatenating, and f_{fusion}( ) is a function of fusion conversion. Since the linear transformation for projection and the function of fusion conversion have been well known to one skilled in the relevant art, detailed explanation of the same is omitted herein for the sake of brevity.

The signaling-pathway component 122 is an artificial neural network having a transformer architecture. For each cell sample related to the biological component, the signaling-pathway component 122 is configured to obtain multi-omics data related to the cell sample, and to convert the multi-omics data into a signaling-pathway feature vector. The multi-omics data may be obtained from public databases such as the cancer dependency map (DepMap) portal, the reactome pathway database, and so on. The signaling-pathway feature vector includes multiple elements that are related respectively to multiple biological signaling pathways all related to the cell sample. In mathematics, the signaling-pathway feature vector can be expressed as H_{c}={p₁, p₂, . . . , p_{M}}, where H_{c} represents the signaling-pathway feature vector, and p₁, p₂, . . . , p_{M} are M number of elements, M is a positive integer and represents a total number of biological signaling pathways. In this embodiment, each of the elements is a real vector, i.e., pᵢ∈R^{D}, i is one of 1, 2 . . . . M, and D represents an embedding dimension and is exemplarily 128 in this embodiment.

Specifically, the multi-omics data contains a gene-expression-level vector that indicates a plurality of gene expression levels respectively of specific genes in the cell sample, and a gene-mutation-state vector that indicates a plurality of gene mutation states respectively of the specific genes in the cell sample. The signaling-pathway component 122 includes a transformer encoder. The signaling-pathway component 122 is configured to obtain the signaling-pathway feature vector by creating a binary matrix that indicates, for each of the specific genes, whether or not the specific gene is involved in each of the specific biological signaling pathways related to the cell sample, calculating a dot product of the binary matrix and the gene-expression-level vector as a first pathway vector, calculating a Hadamard product of the first pathway vector and the gene-mutation-state vector as a second pathway vector, and making the second pathway vector serve as a token and feeding the token into the transformer encoder to obtain the signaling-pathway feature vector, wherein each of the elements of the signaling-pathway feature vector indicates a state of the cell sample in the corresponding one of the specific biological signaling pathways. In particular, the binary matrix has multiple rows corresponding respectively to the biological signaling pathways, and multiple columns corresponding respectively to the specific genes. Each element of the binary matrix has a value of one when the corresponding one of the specific genes is involved in the corresponding one of the specific biological signaling pathways, and has a value of zero when the corresponding one of the specific genes is not involved in the corresponding one of the specific biological signaling pathways.

The interaction-modeling component 123 is configured to obtain the multidimensional vector from the drug-feature integration component 121 and to obtain the signaling-pathway feature vectors that correspond respectively to multiple cell samples related to the biological component from the signaling-pathway component 122. The interaction-modeling component 123 is further configured to determine an interaction vector representing interaction of the specific drug with each of the biological signaling pathways based on the multidimensional vector and the signaling-pathway feature vectors using multi-head attention mechanism. In mathematics, the interaction vector c_{d} can be expressed as c_{d}=Attention(h_{d}, H_{c}), where Attention( ) represents a function of multi-head attention mechanism. Specifically, for each of the signaling-pathway feature vectors, the interaction-modeling component 123 is configured to perform a refinement process with the multidimensional vector serving as a query and the signaling-pathway feature vector serving as a key and a value for calculating attention weights respectively for the biological signaling pathways. The interaction-modeling component 123 is configured to iterate the refinement process for each of the signaling-pathway feature vectors, to calculate the attention weights for each of the signaling-pathway feature vectors based on the queries and the keys, and to calculate the interaction vector based on the attention weights for each of the signaling-pathway feature vectors and the values respectively of the signaling-pathway feature vectors.

The output component 124 is an MLP. The output component 124 is configured to obtain the multidimensional vector from the drug-feature integration component 121 and the interaction vector from the interaction-modeling component 123, and to integrate the multidimensional vector and the interaction vector into a prediction indicator which indicates the drug response of the biological component to the specific drug. In this embodiment, the prediction indicator indicates a value of half maximal inhibitory concentration (IC₅₀), but is not limited thereto. The output component 124 is further configured to determine a dosage of the specific drug to be administrated to a patient with a disease that is attributed to the biological component based on the predication indicator. For example, the greater the predication indicator (i.e., the greater the value of IC₅₀), the greater the dosage of the specific drug to be administrated; the less the predication indicator, the less the dosage of the specific drug to be administrated. Thereafter, a medical professional or a healthcare worker could administrate the specific drug to the patient with the dosage thus determined.

In mathematics, the prediction indicator \hat{r}_{d,c} can be expressed as \hat{r}_{d,c}=f_{pred}([h_{d}∥c_{d}]), where f_{pred} represents a function of linear regression that performs dimensionality reduction on a concatenation of the multidimensional vector and the interaction vector to obtain a real number (i.e., the prediction indicator).

The response prediction module 12 is trained in advanced by using supervised learning techniques with training materials that are labelled with reference indicators (e.g., actual values of IC₅₀) as the ground truth. The training materials may be obtained from public databases like the genomics of drug sensitivity in cancer (GDSC) database, but is not limited thereto.

It should be noted that each of the drug-structure encoder 111 of the feature analysis module 11, the transcriptomic-response decoder 112 of the feature analysis module 11, the drug-structure encoding component 125 of the response prediction module 12, the drug-feature integration component 121 of the response prediction module 12, the signaling-pathway component 122 of the response prediction module 12, the interaction-modeling component 123 of the response prediction module 12 and the output component 124 of the response prediction module 12 may be implemented by one of hardware, firmware, software, and any combination thereof. For example, the drug-structure encoder 111, the transcriptomic-response decoder 112, the drug-structure encoding component 125, the drug-feature integration component 121, the signaling-pathway component 122, the interaction-modeling component 123 and the output component 124 may be implemented to be software modules in a program, where the software modules contain codes and instructions to carry out specific functionalities, and can be called individually or together to fulfill the system 1 of this disclosure.

The above-mentioned modules (i.e., the feature analysis module 11 and the response prediction module 12) may be embodied in: executable software as a set of logic instructions stored in a machine- or computer-readable storage medium of a memory such as RAM, ROM, PROM, firmware, flash memory, etc.; configurable logic such as programmable logic arrays (PLAs), field programmable gate arrays (FPGAs), complex programmable logic devices (CPLDs), etc.; fixed-functionality logic hardware using circuit technology such as application specific integrated circuit (ASIC), complementary metal oxide semiconductor (CMOS), transistor-transistor logic (TTL) technology, etc.; or any combination thereof.

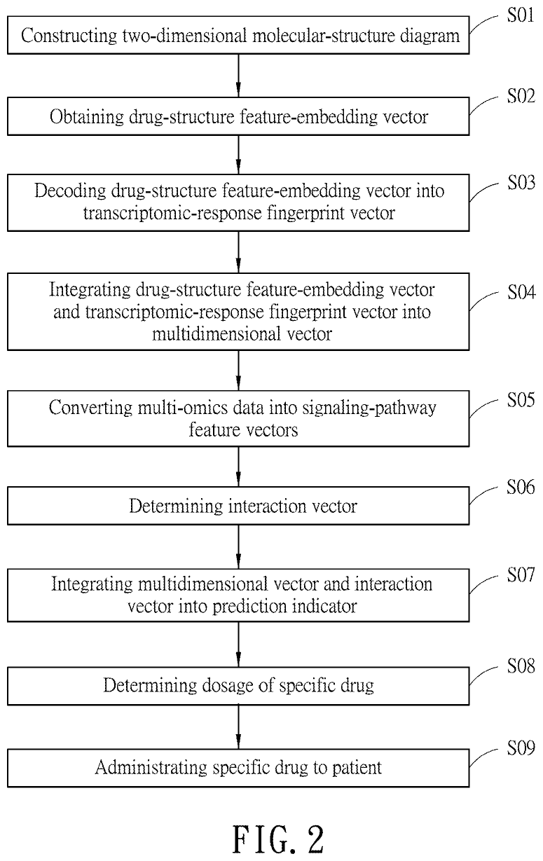

Referring to FIG. 2, an embodiment of a method for predicting a drug response of a biological component to a specific drug according to the disclosure is illustrated. The method is to be implemented by the system 1 that is previously described. The method includes steps S01 to S09 delineated below.

In step S01, the drug-structure encoder 111 constructs the two-dimensional molecular-structure diagram based on the line notation describing the chemical structure of the specific drug.

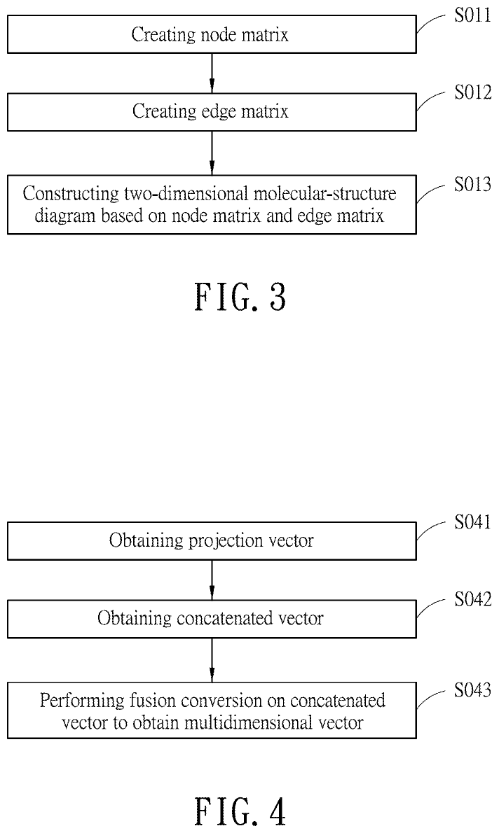

Specifically, step S01 includes sub-steps S011 to S013 that are shown in FIG. 3 and that are delineated below.

In sub-step S011, the drug-structure encoder 111 creates the node matrix for atoms of the molecule of the specific drug by using one-hot encoding, wherein the node matrix is a stack of feature vectors corresponding respectively to the atoms.

In sub-step S012, the drug-structure encoder 111 creates the edge matrix for chemical bonds of the molecule of the specific drug, wherein the edge matrix is a set of indexes each corresponding to one of the chemical bonds between two of the atoms.

In sub-step S013, the drug-structure encoder 111 constructs the two-dimensional molecular-structure diagram based on the node matrix and the edge matrix.

In step S02, the drug-structure encoder 111 performs multi-layer convolution and global add pooling on the two-dimensional molecular-structure diagram so as to obtain the drug-structure feature-embedding vector.

In step S03, the transcriptomic-response decoder 112 decodes the drug-structure feature-embedding vector into the transcriptomic-response fingerprint vector representing transcriptomic response of the biological component to the specific drug.

In step S04, the drug-feature integration component 121 integrates the drug-structure feature-embedding vector and the transcriptomic-response fingerprint vector into the multidimensional vector representing various features of the specific drug.

Specifically, step S04 includes sub-steps S041 to S043 that are shown in FIG. 4 and that are delineated below.

In sub-step S041, the drug-feature integration component 121 projects the transcriptomic-response fingerprint vector to the embedding dimension consistent with the drug-structure feature-embedding vector via linear transformation to obtain the projection vector.

In sub-step S042, the drug-feature integration component 121 concatenates the drug-structure feature-embedding vector and the projection vector into the concatenated vector.

In sub-step S043, the drug-feature integration component 121 performs fusion conversion on the concatenated vector to obtain the multidimensional vector.

In step S05, for each cell sample related to the biological component, the signaling-pathway component 122 obtains the multi-omics data related to the cell sample, and converts the multi-omics data into the signaling-pathway feature vector, wherein the signaling-pathway feature vector includes the elements that are related respectively to the biological signaling pathways all related to the cell sample.

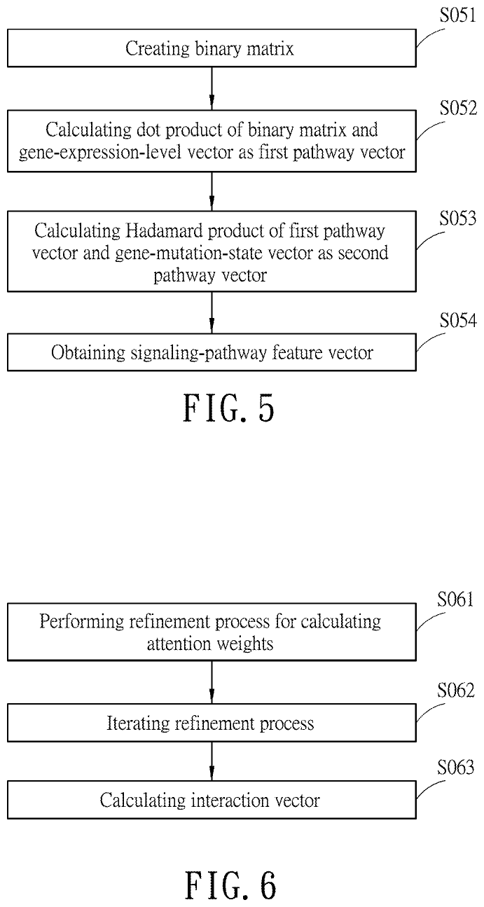

Specifically, step S05 includes sub-steps S051 to S054 that are shown in FIG. 5 and that are delineated below.

In sub-step S051, the signaling-pathway component 122 creates the binary matrix that indicates, for each of the specific genes, whether or not the specific gene is involved in each of the specific biological signaling pathways related to the cell sample.

In sub-step S052, the signaling-pathway component 122 calculates the dot product of the binary matrix and the gene-expression-level vector as the first pathway vector.

In sub-step S053, the signaling-pathway component 122 calculates the Hadamard product of the first pathway vector and the gene-mutation-state vector as the second pathway vector.

In sub-step S054, the signaling-pathway component 122 makes the second pathway vector serve as the token and feeds the token into the transformer encoder to obtain the signaling-pathway feature vector, wherein each of the elements of the signaling-pathway feature vector indicates the state of the cell sample in the corresponding one of the specific biological signaling pathways.

In step S06, the interaction-modeling component 123 determines the interaction vector representing interaction of the specific drug with each of the biological signaling pathways based on the multidimensional vector and the signaling-pathway feature vectors using multi-head attention mechanism.

Specifically, step S06 includes sub-steps S061 to S063 that are shown in FIG. 6 and that are delineated below.

In sub-step S061, for each of the signaling-pathway feature vectors, the interaction-modeling component 123 performs the refinement process with the multidimensional vector serving as the query and the signaling-pathway feature vector serving as the key and the value for calculating attention weights respectively for the biological signaling pathways.

In sub-step S062, the interaction-modeling component 123 iterates the refinement process for each of the signaling-pathway feature vectors.

In sub-step S063, the interaction-modeling component 123 calculates the attention weights respectively for the biological signaling pathways based on the queries and the keys, and calculates the interaction vector based on the attention weights for each of the signaling-pathway feature vectors and the values respectively of the signaling-pathway feature vectors.

In step S07, the output component 124 integrates the multidimensional vector and the interaction vector into the prediction indicator which indicates the drug response of the biological component to the specific drug.

In step S08, the output component 124 determines a dosage of the specific drug to be administrated to a patient with a disease that is attributed to the biological component based on the prediction indicator.

In step S09, the specific drug is administrated to the patient with the dosage thus determined.

In order to evaluate the accuracy and stability of prediction made by using the method and the system 1 according to the disclosure under various combinations of drugs and cell lines (which include cancer cell lines), 5-fold cross-validation was conducted, where a validation index of Pearson correlation coefficient (PCC) is utilized to evaluate a degree of linear correlation between a predicted value of IC₅₀ that is indicated by the prediction indicator outputted by the system 1 and an actual value of IC₅₀ that is obtained by conducting experiments in the real world, and a validation index of root mean square error (RMSE) is utilized to evaluate a degree of deviation of the predicted value of IC₅₀ from the actual value of IC₅₀. A result of the 5-fold cross-validation is shown in Table 1 below.

TABLE 1

| Serial Number of | | |
|---|---|---|
| Validation | PCC | RMSE |
| 1 | 0.87 | 1.42 |
| 2 | 0.87 | 1.47 |
| 3 | 0.87 | 1.45 |
| 4 | 0.87 | 1.45 |
| 5 | 0.87 | 1.45 |
| Average | 0.87 | 1.45 |

Table 1 shows that the validation indexes of PCC all achieve 0.87 and the validation indexes of RMSE are all around 1.45. That is to say, the prediction made by using the method and the system 1 according to the disclosure is relatively accurate and stable under various combinations of drugs and cell lines.

In order to evaluate the predictive ability of the method and the system 1 according to the disclosure, hundreds of cell lines that are unlabeled with any clinical indication or any disease were used to determine, based on the attention weights calculated by the interaction-modeling component 123 in step S06 of the method, which one of the hundreds of cell lines is significantly related to the specific drug of methotrexate (i.e., MTX). It was found that the cell line “JURL-MK1” among the hundreds of cell lines is highly and consistently sensitive to the specific drug of methotrexate according to the attention weights respectively for the biological signaling pathways “RESPONSE_OF_EIF2AK4_GCN2_TO_AMINO_ACIC_DEFICIENCY”, “MITOTIC_METAPHASE_AND_ANAPHASE” and “CELLULAR_RESPONSE_TO_STARVATION” of the cell line “JURL-MK1”. Reference may be made to Table 2 below. External data shows that the cell line “JURL-MK1” is related with chronic myeloid leukemia (CML), which is the target of the specific drug of methotrexate. In particular, methotrexate is a kind of antifolate and inhibits DNA replication by blocking nucleotide synthesis, resulting in cell cycle arrest. Such mechanism of action (MOA) shows that the method and the system according to the disclosure may provide room for interpretation of biological mechanisms. At the same time, it has been verified that the method and the system according to the disclosure may be used to find potential targets simply based on the chemical structure of the specific drug and biological features derived from the multi-omics data.

TABLE 2

| | Attention | | |
|---|---|---|---|
| Biological Signaling Pathway | Weights | Rank | MOA |
| RESPONSE_OF_EIF2AK4_GCN2_{—} | 0.0479 | 1 | Metabolic |
| TO_AMINO_ACIC_DEFICIENCY | | | stress |
| | | | response |
| MITOTIC_METAPHASE_AND_{—} | 0.0429 | 2 | Cell |
| ANAPHASE | | | cycle |
| | | | arrest |
| CELLULAR_RESPONSE_TO_{—} | 0.0426 | 3 | Impaired |
| STARVATION | | | nucleotide |
| | | | synthesis |

To sum up, for the system and the method for predicting a drug response of a biological component to a specific drug according to the disclosure, the drug-structure encoder of the feature analysis module constructs the two-dimensional molecular-structure diagram based on the line notation of the chemical structure of the specific drug, and performs multi-layer convolution and global add pooling on the two-dimensional molecular-structure diagram to obtain the drug-structure feature-embedding vector; the transcriptomic-response decoder of the feature analysis module decodes the drug-structure feature-embedding vector into the transcriptomic-response fingerprint vector; the drug-feature integration component of the response prediction module integrates the drug-structure feature-embedding vector and the transcriptomic-response fingerprint vector into the multidimensional vector; the signaling-pathway component of the response prediction module converts the multi-omics data into the signaling-pathway feature vectors respectively for the cell samples related to the biological component; the interaction-modeling component of the response prediction module determines an interaction vector based on the multidimensional vector and the signaling-pathway feature vectors using multi-head attention mechanism; and the output component of the response prediction module integrates the multidimensional vector and the interaction vector into the prediction indicator which indicates the drug response of the biological component to the specific drug. In this way, a relatively stable and accurate prediction of a drug response may be made for various drugs and various cellular environments in a cross-modal integration way. That is to say, the system and the method according to the disclosure is remarkable for its generalization ability. It is worthy of note that the attention weights determined for the biological signaling pathways show interactions between the specific drug and the biological component, and may provide relevant personnel (e.g., researchers and developers of drugs) with data that has interpretability on biological mechanisms. Consequently, costs of developing drugs may be reduced. At the same time, the fields of pharmaceutical analysis, drug repurposing and precision medicine may be facilitated.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A system for predicting a drug response of a biological component to a specific drug, comprising:
a feature analysis module including
a drug-structure encoder configured to obtain a line notation describing a chemical structure of the specific drug, to construct a two-dimensional molecular-structure diagram based on the line notation of the chemical structure of the specific drug, and to perform multi-layer convolution and global add pooling on the two-dimensional molecular-structure diagram so as to obtain a drug-structure feature-embedding vector, and
a transcriptomic-response decoder configured to obtain the drug-structure feature-embedding vector from said drug-structure encoder, and to decode the drug-structure feature-embedding vector into a transcriptomic-response fingerprint vector representing transcriptomic response of the biological component to the specific drug; and
a response prediction module including
a drug-feature integration component configured to obtain the drug-structure feature-embedding vector, to obtain the transcriptomic-response fingerprint vector from said transcriptomic-response decoder, and to integrate the drug-structure feature-embedding vector and the transcriptomic-response fingerprint vector into a multidimensional vector representing various features of the specific drug,
a signaling-pathway component configured, for each cell sample related to the biological component, to obtain multi-omics data related to the cell sample and to convert the multi-omics data into a signaling-pathway feature vector, the signaling-pathway feature vector including multiple elements that are related respectively to multiple biological signaling pathways all related to the cell sample,
an interaction-modeling component configured to obtain the multidimensional vector from said drug-feature integration component and to obtain the signaling-pathway feature vectors that correspond respectively to multiple cell samples related to the biological component from said signaling-pathway component, and to determine an interaction vector representing interaction of the specific drug with each of the biological signaling pathways based on the multidimensional vector and the signaling-pathway feature vectors using multi-head attention mechanism, and
an output component configured to obtain the multidimensional vector from said drug-feature integration component and the interaction vector from said interaction-modeling component, and to integrate the multidimensional vector and the interaction vector into a prediction indicator which indicates the drug response of the biological component to the specific drug.

2. The system as claimed in claim 1, wherein said output component is further configured to:
determine a dosage of the specific drug to be administrated to a patient with a disease that is attributed to the biological component based on the predication indicator.

3. The system as claimed in claim 1, wherein said drug-structure encoder is configured to construct the two-dimensional molecular-structure diagram by:
creating a node matrix for atoms of a molecule of the specific drug by using one-hot encoding, the node matrix being a stack of feature vectors corresponding respectively to the atoms;
creating an edge matrix for chemical bonds of the molecule of the specific drug, the edge matrix being a set of indexes each corresponding to one of the chemical bonds between two of the atoms; and
constructing the two-dimensional molecular-structure diagram based on the node matrix and the edge matrix.

4. The system as claimed in claim 1, wherein said drug-feature integration component is configured to obtain the multidimensional vector by projecting the transcriptomic-response fingerprint vector to an embedding dimension consistent with the drug-structure feature-embedding vector via linear transformation to obtain a projection vector, concatenating the drug-structure feature-embedding vector and the projection vector into a concatenated vector, and performing fusion conversion on the concatenated vector to obtain the multidimensional vector.

5. The system as claimed in claim 1, wherein the multi-omics data contains a gene-expression-level vector that indicates a plurality of gene expression levels respectively of specific genes in the cell sample, and a gene-mutation-state vector that indicates a plurality of gene mutation states respectively of the specific genes in the cell sample,
wherein said signaling-pathway component includes a transformer encoder, and is configured to obtain the signaling-pathway feature vector by
creating a binary matrix that indicates, for each of the specific genes, whether or not the specific gene is involved in each of the specific biological signaling pathways related to the cell sample,
calculating a dot product of the binary matrix and the gene-expression-level vector as a first pathway vector,
calculating a Hadamard product of the first pathway vector and the gene-mutation-state vector as a second pathway vector, and
making the second pathway vector serve as a token and feeding the token into the transformer encoder to obtain the signaling-pathway feature vector, each of the elements of the signaling-pathway feature vector indicating a state of the cell sample in the corresponding one of the specific biological signaling pathways.

6. The system as claimed in claim 1, wherein said interaction-modeling component is configured to obtain the interaction vector by:
for each of the signaling-pathway feature vectors, performing a refinement process with the multidimensional vector serving as a query and the signaling-pathway feature vector serving as a key and a value for calculating attention weights respectively for the biological signaling pathways;
iterating the refinement process for each of the signaling-pathway feature vectors; and
calculating the interaction vector based on the attention weights for each of the signaling-pathway feature vectors and the values respectively of the signaling-pathway feature vectors.

7. The system as claimed in claim 1, wherein said response prediction module further includes a drug-structure encoding component that is configured to obtain the line notation describing the chemical structure of the specific drug, to construct the two-dimensional molecular-structure diagram based on the line notation describing the chemical structure of the specific drug, and to perform multi-layer convolution and global add pooling on the two-dimensional molecular-structure diagram so as to obtain the drug-structure feature-embedding vector.

8. The system as claimed in claim 1, wherein said drug-structure encoder is a graph isomorphism network (GIN).

9. The system as claimed in claim 1, wherein said transcriptomic-response decoder is a multilayer perceptron (MLP).

10. The system as claimed in claim 1, wherein said signaling-pathway component is an artificial neural network having a transformer architecture.

11. The system as claimed in claim 1, wherein the line notation of the chemical structure of the specific drug follows a specification of simplified molecular input line entry system (SMILES).

12. The system as claimed in claim 1, wherein the prediction indicator indicates a value of half maximal inhibitory concentration (IC₅₀).

13. The system as claimed in claim 1, wherein said output component is a multilayer perceptron (MLP).

14. The system as claimed in claim 1, wherein said response prediction module is a multimodal neural network.

15. A method for predicting a drug response of a biological component to a specific drug, to be implemented by a system that includes a drug-structure encoder, a transcriptomic-response decoder, a drug-feature integration component, a signaling-pathway component, an interaction-modeling component and an output component, the method comprising steps of:
constructing, by the drug-structure encoder, a two-dimensional molecular-structure diagram based on a line notation describing a chemical structure of the specific drug;
performing, by the drug-structure encoder, multi-layer convolution and global add pooling on the two-dimensional molecular-structure diagram so as to obtain a drug-structure feature-embedding vector;
decoding, by the transcriptomic-response decoder, the drug-structure feature-embedding vector into a transcriptomic-response fingerprint vector representing transcriptomic response of the biological component to the specific drug;
integrating, by the drug-feature integration component, the drug-structure feature-embedding vector and the transcriptomic-response fingerprint vector into a multidimensional vector representing various features of the specific drug;
for each cell sample related to the biological component by the signaling-pathway component,
obtaining multi-omics data related to the cell sample,
converting the multi-omics data into a signaling-pathway feature vector, the signaling-pathway feature vector including multiple elements that are related respectively to multiple biological signaling pathways all related to the cell sample;
determining, by the interaction-modeling component, an interaction vector representing interaction of the specific drug with each of the biological signaling pathways based on the multidimensional vector and the signaling-pathway feature vectors using multi-head attention mechanism; and
integrating, by the output component, the multidimensional vector and the interaction vector into a prediction indicator which indicates the drug response of the biological component to the specific drug.

16. The method as claimed in claim 15, further comprising steps of:
determining a dosage of the specific drug to be administrated to a patient with a disease that is attributed to the biological component based on the prediction indicator; and
administrating the specific drug to the patient with the dosage thus determined.

17. The method as claimed in claim 15, wherein the step of constructing the two-dimensional molecular-structure diagram includes:
creating a node matrix for atoms of a molecule of the specific drug by using one-hot encoding, the node matrix being a stack of feature vectors corresponding respectively to the atoms;
creating an edge matrix for chemical bonds of the molecule of the specific drug, the edge matrix being a set of indexes each corresponding to one of the chemical bonds between two of the atoms; and
constructing the two-dimensional molecular-structure diagram based on the node matrix and the edge matrix.

18. The method as claimed in claim 15, wherein the step of integrating the drug-structure feature-embedding vector and the transcriptomic-response fingerprint vector includes:
projecting the transcriptomic-response fingerprint vector to an embedding dimension consistent with the drug-structure feature-embedding vector via linear transformation to obtain a projection vector;
concatenating the drug-structure feature-embedding vector and the projection vector into a concatenated vector; and
performing fusion conversion on the concatenated vector to obtain the multidimensional vector.

19. The method as claimed in claim 15, wherein the multi-omics data contains a gene-expression-level vector that indicates a plurality of gene expression levels respectively of specific genes in the cell sample, and a gene-mutation-state vector that indicates a plurality of gene mutation states respectively of the specific genes in the cell sample,
wherein the signaling-pathway component includes a transformer encoder, and
wherein the step of converting the multi-omics data into the signaling-pathway feature vector includes:
creating a binary matrix that indicates, for each of the specific genes, whether or not the specific gene is involved in each of the specific biological signaling pathways related to the cell sample;
calculating a dot product of the binary matrix and the gene-expression-level vector as a first pathway vector;
calculating a Hadamard product of the first pathway vector and the gene-mutation-state vector as a second pathway vector; and
making the second pathway vector serve as a token and feeding the token into the transformer encoder to obtain the signaling-pathway feature vector, each of the elements of the signaling-pathway feature vector indicating a state of the cell sample in the corresponding one of the specific biological signaling pathways.

20. The method as claimed in claim 15, wherein the step of determining the interaction vector includes:
for each of the signaling-pathway feature vectors, performing a refinement process with the multidimensional vector serving as a query and the signaling-pathway feature vector serving as a key and a value for calculating attention weights respectively for the biological signaling pathways;
iterating the refinement process for each of the signaling-pathway feature vectors; and
calculating the interaction vector based on the attention weights for each of the signaling-pathway feature vectors and the values respectively of the signaling-pathway feature vectors.
